(19) United States
(12) Patent Application Publication
(10) Pub. No.: US 2024/0189237 A1
(43) Pub. Date: Jun. 13, 2024

(54) FORMULATION AND METHOD FOR TREATMENT OF URINARY SYSTEM DISORDERS

(71) Applicant: WATERSHED MEDICAL INC, Bellevue, WA (US)

(72) Inventors: Christopher R. KINSELLA, Jr., Los Altos, CA (US); Saniya ALI, Seattle, WA (US); Clay Justin NOLAN, Morgan Hill, CA (US); Rush Lloyd BARTLETT, II, Austin (CA)

(73) Assignee: WATERSHED MEDICAL INC, Bellevue, WA (US)

(21) Appl. No.: 18/285,645

(22) PCT Filed: Apr. 7, 2021
(86) PCT No.: PCT/US21/26285

§ 371 Date: Oct. 4, 2023

(51) Int. Cl. A61K 9/16; A61K 9/00; A61K 31/546; A61K 31/555; A61P 31/00

(52) U.S. Cl. CPC A61K 9/1676; A61K 9/0034; A61K 9/1617; A61K 9/1641; A61K 31/546; A61K 31/555; A61P 31/00

(57) ABSTRACT

The invention provides a formulation and method for the treatment of urinary system disorders, wherein the formulation comprises a controlled release drug delivery system for intravesicular administration to a patient. Controlled release microparticles in the formulation are comprised of a pharmacologically active agent and a controlled release carrier, where the microparticles are buoyant in urine. Microparticle buoyancy in urine ensures that the formulation continues to release the active agent into a patient's bladder throughout the duration of an extended drug delivery time period. Methods of treating urinary system disorders by intravesicular administration of the microparticle formulation to a subject are also provided, and include methods of treating urinary system infections, bladder cancer, incontinence, and other urinary system disorders.

### TECHNICAL FIELD

The present invention relates generally to the treatment of urinary system disorders, and, more particularly, relates to a formulation and method for the intravesical administration of a pharmacologically active agent to treat or prevent a disorder of the urinary system.

### BACKGROUND

Treatment of patients suffering from a urinary system disorder is conventionally done via oral drug delivery. Oral administration of a pharmacologically active agent is often a less attractive option than local drug delivery, insofar as higher dosages are required than with local administration, and adverse effects are more likely. The possibility of treating a patient with a urinary system disorder by locally delivering an active agent to the bladder has been explored, but the possibility of “intravesicular” (or “intravesical”) administration to the bladder via the urethra remains problematic. Any pharmaceutical formulation administered to the bladder, even a controlled release formulation intended to provide extended drug release, is eliminated from the bladder every time the bladder is emptied. The present invention is addressed to the aforementioned limitation in art and provides a therapeutically effective formulation and method for intravesicular drug delivery to treat a patient suffering from a urinary system disorder.

### SUMMARY OF THE INVENTION

Accordingly, the invention provides a formulation for intravesicular administration to a patient that achieves a controlled release profile, enabling delivery of an active agent to a patient throughout an extended drug delivery time period.

In one embodiment, a controlled release pharmaceutical formulation is provided for intravesicular administration to a subject, the formulation comprising a primary population of microparticles having a mean diameter in the range of 500 nm to 2000 μm and comprised of 2.5 wt. % to 95 wt. % of a pharmacologically active agent and 5 wt. % to 97.5 wt. % of a controlled release carrier, wherein the active agent, carrier, and relative amounts of the active agent and carrier in the microparticles are effective to render the primary microparticles buoyant in urine. In one aspect, the carrier provides for controlled release of the pharmacologically active agent in the urinary system. In another aspect, the carrier provides for controlled release of the pharmacologically active agent in the bladder. In another aspect, the pharmacologically active agent is a drug for the treatment of a disorder of the urinary system.

In another aspect of the embodiment, the controlled release carrier comprises a matrix and the pharmacologically active agent is dispersed therein. In a different aspect of the embodiment, the microparticles are coated core-type controlled release dosage forms, wherein the controlled release carrier coats an active agent-containing core or wherein an active agent coating encloses a core of the controlled release carrier.

In another aspect, the controlled release carrier is selected so that it gradually dissolves, degrades, or erodes in urine to release the pharmacologically active agent from the microparticles in the bladder.

In some aspects, the carrier comprises a fatty acid, fatty alcohol, fatty acid ester, phospholipid, sterol, polyethylene glycol alkyl ether, polyoxyethylene-polyoxypropylene block copolymer, chitosan, or bile salt. Fatty acid esters typically comprise a lower alcohol fatty acid ester, a triglyceride, a monoglyceride, a triglyceride, a polyglycerized fatty acid, a propylene glycol fatty acid ester, a polyethoxylated fatty acid, a polyethoxylated glyceryl fatty acid ester, a sorbitan fatty acid ester, or a hydroxyacid diester.

In some aspects, the carrier comprises a surfactant selected from sodium lauryl sulfate, cetyltrimethyl-ammonium bromide, benzalkonium chloride, 2-phenoxyethanol, and benzoyl alcohol.

In some aspects, the pharmacologically active agent is selected from anti-infective agents, including anti-bacterial, anti-fungal, and anti-viral agents; chemotherapeutic agents; anti-inflammatory agents; anesthetic agents; analgesic agents; diuretic agents; coagulants and anti-coagulants; biologics; agents for treatment of incontinence, including overactive bladder (including antimuscarinic agents, β3-adrenergic receptor agonists, anesthetic agents, and analgesic agents); renin-angiotensin-aldosterone system (RAAS) inhibitors; agents for treating kidney stones; and contrast agents for diagnostics and monitoring.

In another aspect of the embodiment, subsequent to intravesicular administration of the formulation, the primary population of microparticles takes on a changeable formulation shape within the bladder and adapts to bladder shape as bladder shape changes.

In another embodiment, the formulation additionally includes a secondary population of microparticles that differs from the primary population of microparticles in at least one aspect, such as comprising a different pharmacologically active agent, a different amount of a pharmacologically active agent, a different controlled release carrier, or a different amount of controlled release carrier, or having a different controlled release profile or specific gravity.

In another embodiment, the invention provides a controlled release pharmaceutical formulation for intravesicular administration to a subject to treat a bacterial infection of the bladder, the formulation comprising:

a population of microparticles having a mean diameter in the range of 90 mm to 900 mm and a mean specific gravity of less than 1.005, wherein the microparticles comprise:

50 wt. % to 95 wt. % of a controlled release carrier matrix comprising a C₆-C₂₂ fatty alcohol, a C₆-C₂₂ fatty acid, or a mono-, di- or triester of a C₆-C₂₂ fatty acid; and

5 wt. % to 50 wt. % of silver sulfadiazine dispersed within the carrier matrix.

In another embodiment, a method is provided for treating a urinary system disorder, wherein the method comprises administering a formulation comprised of a primary population of microparticles having a mean diameter in the range of 50 nm to 2000 μm, 2.5 wt. % to 95 wt. % of a pharmacologically active agent, and 5 wt. % to 97.5 wt. % of a controlled release carrier, wherein the active agent, carrier, and relative amounts of the active agent and carrier in the microparticles are effective to render the microparticles buoyant in urine, and further wherein administration is carried out via intravesicular delivery so that the formulation (a) is at least partially retained by the bladder for the duration of a drug delivery time period, and (b) provides controlled release of the active agent in the bladder during the drug delivery time period.

In one aspect of the embodiment, the urinary system disorder comprises a disorder of the bladder, ureters, urethra, kidney, or combination thereof.

In one aspect of the embodiment, the urinary system disorder comprises a urinary system infection (commonly referred to as a “UTI” or urinary tract infection”) such as a bacterial, fungal, or viral infection; a cancer or benign tumor of the urinary system; urinary incontinence (including stress incontinence and overactive bladder, “OAB”) and urinary retention; urinary system inflammation, injury, or scarring; bladder pain syndrome; neurogenic bladder dysfunction; vesicoureteral reflux; a sexually transmitted disease such as chlamydia, gonorrhea, or syphilis; a bladder calculus; or a kidney stone, diabetic nephropathy, pyelonephritis, or kidney failure.

In a further embodiment of the invention, a packaged pharmaceutical formulation is provided, comprising a sealed container housing the microparticle formulation of the invention and instructions for intravesicular administration of the formulation to treat a urinary system disorder in a subject.

In another embodiment, the invention provides a method for making the microparticle formulation of the invention, where the method comprises the following steps: melting or otherwise liquefying a controlled release carrier; adding a pharmacologically active agent for treating a urinary system disorder to the melted carrier to provide an active-agent carrier admixture; homogenizing the admixture to provide a suspension of the active agent in the melted carrier; atomizing the suspension to provide the microparticles as congealed droplets comprised of the active agent and carrier; and collecting the microparticles to provide the formulation.

### BRIEF DESCRIPTION OF THE DRAWINGS

FIG. 1 schematically illustrates the method for making microparticles as described in Example 4.

FIG. 2 (Table 3) sets forth the results of the evaluation of antimicrobial efficacy of silver sulfadiazine (SSD) and cefpodoxime as described in Example 2.

FIG. 3 is a graph illustrating the percentage of SSD released over time from microparticles of the invention as determined in Example 5. As discussed in the example, the results indicate that a zero order release profile was achieved.

### DETAILED DESCRIPTION OF THE INVENTION

### 1. Definitions and Overview

Unless defined otherwise, all technical and scientific terms used herein have the meaning commonly understood by one of ordinary skill in the art to which the invention pertains. Specific terminology of particular importance to the description of the present invention is defined below.

In this specification and the appended claims, the singular forms “a,” “an” and “the” include plural referents unless the context clearly dictates otherwise. Thus, for example, reference to “a pharmacologically active agent” or simply “an active agent” includes a single such agent as well as two or more such agents; “a pharmaceutically acceptable carrier” refers to a combination of pharmaceutically acceptable carriers as well as to a single pharmaceutically acceptable carrier; “a formulation” and “a vehicle” includes two or more formulations and vehicles, respectively, and the like.

The terms “active agent,” “pharmacologically active agent” and “drug” are used interchangeably herein to refer to a chemical compound that induces a desired pharmacological, physiological effect. The aforementioned terms also encompass pharmaceutically acceptable, pharmacologically active derivatives of those active agents specifically mentioned herein, including, but not limited to, salts, esters, amides, prodrugs, active metabolites, conjugates, analogs, and the like. Accordingly, when referring to an active agent, whether specified as a particular compound (e.g., silver sulfadiazine) or a compound class (e.g., antibacterial agent), the term used to refer to the agent is intended to encompass not only the specified molecular entity but also its pharmaceutically acceptable, pharmacologically active analogs and derivatives, including, but not limited to, salts, esters, amides, prodrugs, conjugates, active metabolites, hydrates, crystalline forms, enantiomers, stereoisomers, and other such derivatives, analogs, and related compounds.

An “analog” of a pharmacologically active agent refers to a compound having the same type of pharmacological activity as the parent compound and approximately equivalent in degree.

By “pharmaceutically acceptable” is meant a material that is not biologically or otherwise undesirable, i.e., the material may be incorporated into a pharmaceutical composition as provided herein and not cause any substantial undesirable biological effects or interact in a deleterious manner with any of the other components of the composition. When the term “pharmaceutically acceptable” is used to refer to a solid or semi-solid carrier, liquid vehicle, or other excipient, i.e., to any inactive ingredient herein, it is implied that the carrier or excipient has met the required standards of toxicological and manufacturing testing and/or that it is included on the Inactive Ingredient Guide prepared by the U.S. Food and Drug Administration and designated “Generally Regarded as Safe” (“GRAS”).

The term “controlled release” refers to a mechanism of drug delivery wherein administration of an active agent-containing formulation or fraction thereof does not result in the immediate release of 100% of the active agent. The term is used interchangeably with “nonimmediate release” as defined in Remington: The Science and Practice of Pharmacy, Nineteenth Ed. (Easton, PA: Mack Publishing Company, 1995). In general, the term “controlled release” as used herein includes sustained release, modified release, pulsatile release, and delayed release formulations, as well as formulations that combine two or more types of release profiles, such as immediate release of a bolus dose followed by pulsatile release or sustained release thereafter. “Sustained release” (synonymous with “extended release”) refers to a formulation that provides for gradual release of an active agent over an extended period of time, and that preferably, although not necessarily, results in substantially constant levels of an agent in a volume of distribution (e.g., blood, urine, bladder, total body water an extended time period, etc.), and is normally referred to as “zero order” release. “Controlled release” also includes “delayed release,” indicating a formulation that, following administration to a patient, provides for a measurable time delay before the active agent is released from the formulation into the patient's body. Controlled release dosage forms herein, however, are generally of the sustained release type.

The “microparticles” herein that are formulated so as to contain a pharmacologically active agent may be substantially spherical, or they may be cylindrical or rod-like or having any other shape; the term “microparticle” is not limited in this regard. Microparticle size is given as the mean microparticle diameter in a population of microparticles. The microparticle size distribution in population of microparticles is relatively narrow, i.e., the specified mean microparticle size is associated with a fairly low standard of deviation, typically less than 30%, e.g., less than 20% or less than 15%.

The terms “effective amount” and “therapeutically effective amount” of an active agent, an active agent combination, or a pharmaceutical formulation refer to an amount or concentration that is nontoxic but sufficient for producing a desired result. The exact amount required will vary from subject to subject, depending on factors such as the age, weight and general condition of the subject, the particular condition being treated, the severity of the condition, the specific active agent, and the judgment of the clinician.

The terms “treating,” “treatment,” and “therapeutic” as used herein refer to the administration of a pharmacologically active agent to a subject to provide a desired pharmacological or physiological effect, and thus encompasses administration for therapeutic and/or prophylactic purposes. Treating a condition in a subject already suffering from that condition generally involves a reduction in the severity, number, and/or frequency of symptoms, the elimination of symptoms and/or underlying cause, and the improvement or remediation of damage. In the prophylactic context, treatment refers to the administration of a pharmaceutical agent or composition to a subject who is not yet suffering from a particular condition, but has been identified as at susceptible to, i.e., at risk for developing, the particular condition, where the prophylactic effect involves partially or completely preventing a condition or symptom thereof.

The term “urinary system” is used in the conventional sense to refer to the lower urinary tract as well as the upper urinary tract, and thus includes the bladder, urethra, kidneys, and ureters.

The term “disorder” refers to a physiological condition of clinical relevance and thus includes symptomatic or asymptomatic conditions regardless of etiology. Disorders thus include adverse conditions resulting from disease or injury. The disorders addressed with the present invention are disorders of the urinary system.

As used herein, a “subject” or “individual” or “patient” refers to any subject for whom therapy is desired, and generally refers to the recipient of the therapy to be practiced according to the invention.

A “drug delivery time period” refers to a period of time during which a pharmacologically active agent is released from a formulation or fraction thereof, generally an extended time period.

The term “substantially” indicates the possibility of slight deviation from a recited chemical or physical property, and allows for a difference of at most about 20%, or at most about 10%, or at most about 5%, between an actual chemical or physical property and the recited chemical or physical property. The term “substantially homogeneous,” for example, refers to a material in the form of a mixture of two or more components in which the material is substantially uniform throughout, with any two discrete regions within the material differing by at most about 20%, or at most about 10%, or at most about 5%, with respect to a chemical or physical property of the material, such as the presence or absence of a component, the concentration of a component, the degree of hydrophilicity or lipophilicity, density, crystallinity, or the like. Similarly, the term “approximately” in any context is intended to connote a possible variation of at most about 20%. Generally, the term connotes a possible variation of at most about 10%, or at most about 5%.

The invention provides a formulation and method for the controlled release of a pharmacologically active agent within the bladder of a subject to treat a disorder of the urinary system. A formulation that comprises active agent-containing microparticles is administered to the subject via the intravesicular route, i.e., through the urethra or via a bladder injection, wherein the microparticles are buoyant in urine and therefore float to the surface of urine present in the bladder. Upon voiding, the bladder contracts and the thick, irregular mucosal tissue known as rugae, located on the interior walls of the bladder, traps the microparticles in the resulting folds, so that release of active agent within the bladder continues over a series of cycles in which the bladder refills and empties with urine. Furthermore, particle size, degradation or aggregation of microparticles, and other factors can be varied to facilitate retention within the bladder without necessarily requiring that microparticles be trapped in the rugae as the bladder contracts. Any of these retention means can be used in combination, and some may exhibit synergy with respect to enhancing the mechanistic process of an alternative retention means.

In addition, contact between the microparticles and the interior walls of the bladder or urethra, as well as microparticle movement within the bladder causing turbulence in urine, substantially prevents biofilm formation and facilitates mechanical disruption of any biofilm already present, in turn reducing the likelihood of adherence of bacterial cells to a biofilm matrix. By disrupting biofilms in the bladder and urethra, then, the formulation and method of the invention can minimize or eliminate bacteria growth within the bladder and urethra.

As explained in co-pending, commonly assigned U.S. patent application Ser. No. 17/062,469, filed Oct. 2, 2020, and International Patent Publication No. WO 2020/069,376 A1, the invention avoids the need for oral medication and systemic dosing to treat disorders of the urinary system, and provides numerous advantages in treatment of such disorders. The aforementioned patent applications are incorporated herein by reference in their entireties, and reference may be had thereto for additional information on the properties, components, and uses of the present microparticle formulation.

### 2. Microparticles

The microparticles within the pharmaceutical formulation of the invention are comprised of a pharmacologically active agent and a controlled release carrier, which by gradual reduction in mass and/or other means releases the pharmacologically active agent from the microparticles into the bladder during an extended drug delivery time period.

### A. Active Agents:

The choice of pharmacologically active agent is, of course, dependent on the indication being treated. Active agents that are administrable via intravesicular administration of a microparticle formulation of the invention are generally, although not necessarily, selected from the following categories: anti-infective agents, including anti-bacterial, anti-fungal, and anti-viral agents; chemotherapeutic agents; anti-inflammatory agents; anesthetic agents; analgesic agents; diuretic agents; coagulants and anti-coagulants; biologics; agents for treatment of incontinence, including overactive bladder (including antimuscarinic agents, β3-adrenergic receptor agonists, anesthetic agents, and analgesic agents); renin-angiotensin-aldosterone system (RAAS) inhibitors; agents for treating kidney stones; and contrast agents for diagnostics and monitoring. Specific examples of active agents that can be administered to treat a urinary system disorder as provided herein are as follows:

### Anti-Infective Agents:

tetracycline antibiotics and related compounds (e.g., chlortetracycline, oxy-tetracycline, demeclocycline, methacycline, doxycycline, minocycline and roli-tetracycline);
macrolide antibiotics such as erythromycin, clarithromycin, and azithromycin;
streptogramin antibiotics such as quinupristin and dalfopristin;
beta-lactam antibiotics, including penicillins (e.g., penicillin G, penicillin VK), antistaphylococcal penicillins (e.g. cloxacillin, dicloxacillin, nafcillin and oxacillin), extended spectrum penicillins (e.g. aminopenicillins such as ampicillin, amoxicillin, and benzathine benzylpenicillin, and antipseudomonal penicillins such as carbenicillin), cephalosporins (e.g. cefadroxil, cefepime, cefpodoxime, cephalexin, cefazolin, cefoxitin, cefotetan, cefuroxime, cefotaxime, ceftazidime and ceftriaxone) and carbapenems such as imipenem, meropenem and aztreonam;
aminoglycoside antibiotics such as streptomycin, gentamicin, tobramycin, amikacin and neomycin;
glycopeptide antibiotics such as teicoplanin;
sulfonamide antibiotics such as sulfacetamide, sulfabenzamide, sulfadiazine, sulfadoxine, sulfamerazine, sulfamethazine, sulfamethizole and sulfamethoxazole;
quinolone antibiotics such as levofloxacin, trovafloxacin, ciprofloxacin, nalidixic acid and ofloxacin;
anti-mycobacterials such as isoniazid, rifampin, rifabutin, ethambutol, pyrazinamide, ethionamide, aminosalicylic and cycloserine;
nitrofurans such as nitrofurazone and nitrofurantoin;
antifungal agents such as itraconazole, ketoconazole, fluconazole and amphotericin B;
miscellaneous antimicrobial agents such as trimethoprim, chloramphenicol, fosfomycin, spectinomycin, polymyxin B (colistin), bacitracin, nitrofurantoin, methenamine, and sodium oxychlorosene.

Agents for treating viral infections of the urinary system include, by way of example, anti-herpes agents such as aciclovir, famciclovir, foscarnet, ganciclovir, idoxuridine, sorivudine, trifluridine, valacyclovir and vidarabine; anti-retroviral agents such as didanosine, stavudine, zalcitabine, tenovovir and zidovudine; other antiviral agents including amantadine, interferon-α, ribavirin and rimantadine. Of particular interest for treatment of viral infections of the urinary system are cidofovir and leflunomide.

Antibacterial agents of particular interest herein are oligodynamic active agents, i.e., biocidal metal-based agents. Many such agents are silver-based, and include elemental silver, silver ions, silver salts, and silver coordination compounds. Silver salts can be inorganic, such as silver bromide, silver chloride, silver iodate, silver iodide, silver nitrate, silver oxide, silver perchlorate, and silver tetrafluoroborate. Organic silver-containing compounds include both organic silver salts and coordination compounds, for instance silver acetate, silver benzoate, silver carbonate, silver lactate, silver laurate, silver palmitate, and silver sulfadiazine (SSD). Other metals, such as gold, zinc, copper, and cerium, have also been found to possess antimicrobial properties, both alone and in combination with silver.

The selection of an anti-infective agent in any particular case will depend on the nature of the infective agent as well as other factors. Urinary system infections are commonly caused by the microorganisms Escherichia coli (E. coli), Klebsiella pneumonia, Staphylococcus saprophyticus, Proteus mirabilis, Enterococcus faecalis, Staphylococcus aureus, Candida albicans, Streptococcus agalactiae, Mycoplasma genitalium, Pseudomonas aeruginosa, Chlamydia trachomatis, and the herpes simplex viruses Human alphaherpesvirus 1 (HSV-1) and Human alphaherpesvirus 2 (HSV-2).

### Chemotherapeutic Agents:

Apaziquone; aldesleukin; Bacillus Calmette-Guérin (BCG) immunotherapy; cisplatin; docetaxel; doxorubicin; erdafitinib; everolimus; fosfomycin; gemcitabine; methotrexate; mitomycin C; mitoxantrone; paclitaxel, thiotepa; camptothecin and its analogues and derivatives (e.g. 9-aminocamptothecin, 9-nitrocamptothecin, 10-hydroxycamptothecin, irinotecan, meglumine, topotecan and 20-O-β-glucopyranosyl camptothecin); taxanes (e.g. baccatins, cephalomannine and their derivatives); carboplatin; interleukin (IL)-2 and IL-12; interferon α-2a, interferon α-2b, interferon α-n3 and other agents of the interferon family; levamisole; altretamine; cladribine; tretinoin; procarbazine; dacarbazine; mitotane; asparaginase; porfimer; amifostine; mitotic inhibitors including podophyllotoxin derivatives teniposide and etoposide; and the vinca-alkaloids vinorelbine, vincristine and vinblastine. Of particular interest for the treatment of cancers of the urinary system, including bladder cancers, renal cancers, and cancers of the urethra and ureters include, without limitation, apaziquone, aldesleukin, axitinib, BCG immunotherapy, cisplatin, doxorubicin, erdafitinib, everolimus, fosfomycin, gemcitabine, IL-2, IL-12, methotrexate, mitomycin-C, thiotepa, vinblastine, and the monoclonal antibody medications bevacizumab (Avastin®), avelumab (Bavencio®), cabozantinib-S-malate, ipilimumab, nivolumab, sunitinib malate, and pembrolizumab, among others.

### Anti-Inflammatory Agents:

These include nonsteroidal anti-inflammatory agents (NSAIDs) such as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen, apazone, diclofenac, difenpiramide, diflunisal, etodolac, indomethacin, ketorolac, meclofenamate, nabumetone, phenylbutazone, piroxicam, sulindac and tolmetin; COX-2 inhibitors such as celecoxib, rofecoxib, and valdecoxib; and steroidal anti-inflammatory agents, e.g., hydrocortisone, hydrocortisone-21-monoesters (e.g. hydrocortisone-21-acetate, hydrocortisone-21-butyrate, hydrocortisone-21-propionate, hydrocortisone-21-valerate), hydrocortisone-17,21-diesters (e.g. hydrocortisone-17,21-diacetate, hydrocortisone-17-acetate-21-butyrate, hydrocortisone-17,21-dibutyrate), alclometasone, betamethasone, dexamethasone, flumethasone, prednisolone methylprednisolone, and triamcinolone.

Anti-inflammatory agents of particular interest in the treatment of urinary system inflammation are cromolyn sodium, pentosan polysulfate sodium, and the glycosaminoglycans chondroitin sulfate, hyaluronic acid, and heparin.

### Anesthetic Agents:

Lidocaine, bupivacaine, benzocaine, acetocaine, tetracaine, and prilocaine, among others.

### Analgesic Agents:

These include nonsteroidal analgesic agents such as acetaminophen, acetylsalicylic acid, and the anti-inflammatory agents above, as well as opioid analgesics such as buprenorphine, butorphanol, codeine, diamorphine, dihydrocodeine, ethylmorphine, fentanyl, hydrocodone, hydromorphone, isomethadone, levorphanol, lofentanil, meperidine, methadone, morphine, nalbuphine, oxycodone, oxymorphone, pentazocine, propoxyphene, sufentanil and tramadol. As known in the art, there are opioid receptors in bladder tissue (Westerling et al. (2007), “Opioids and Bladder Pain/Function,” in Schmidt et al. (eds.), Encyclopedia of Pain (Springer, Berlin)), and these agents are therefore useful to treat bladder pain syndrome (BPS) and other pain within the urinary system, regardless of the underlying etiology, using the formulation and method of the invention.

### Diuretic Agents:

These include loop diuretics such as furosemide, ethacrynic acid, bumetanide, and torasemide; thiazide diuretics such as hydrochlorothiazide and bendroflumethiazide; potassium-sparing diuretics such as spironolactone, epierenone, potassium canreonate, amiloride, and triamterene; and xanthenes such as theophylline and theobromine.

### Biologics:

Biological agents administrable according to the invention include, without limitation, proteins, peptides, peptide fragments, monoclonal antibodies, enzymes, amino acids, nucleic acids (e.g., DNA, modified DNA, mRNA), cytokines, hormones, and chimeric antigen receptor (CAR) T cell therapy.

RAAS inhibitors for treatment of diabetic nephropathy include angiotensin-converting-enzyme (ACE) inhibitors such as captopril, zofenopril, fosinopril, enalapril, lisinopril, benazepril, and imidapril; antimuscarinic agents such as darifenacin, hyoscyamine, oxybutynin, tolterodine, solifenacin, trospium, fesoteridine, benztropine mesylate, orphenadrine, procyclidine, and trihexyphenidyl; and β3-adrenergic receptor agonists such as amibegron, mirabegron, nebivolol, solabegron, and vibegron.

Other active agent groups include bladder relaxant drugs, e.g., for treating incontinence resulting from detrusor muscle overactivity (e.g., oxybutynin, ipratropium, and tricyclic antidepressants such as amitriptyline, imipramine and desipramine; capsaicin, baclofen and other GABAB receptor agonists); and drugs for treating incontinence due to neurologic sphincter deficiency (such as α-adrenergic agonists, β-adrenergic agents, estrogenic agents, and tricyclic antidepressants).

Any of the aforementioned active agents and active agent types can be administered in combination, and any such combinations can be particularly advantageous for the treatment of certain urinary system disorders. For instance, in the treatment of urinary tract infections, intravesicular co-administration of an anti-infective agent with an anesthetic agent would both treat the infection and reduce pain. As another example, treatment of a bladder cancer associated with inflammation would benefit from intravesicular co-administration of a chemotherapeutic agent with an anti-inflammatory agent. Additional examples include: administration of an anti-infective agent in combination with delivery of healthy urinary bacteria such as Lactobacillus to treat urinary tract infections; administration of a secondary agent that acts as an oxidizer to increase the potency of the primary active agent, particularly when the primary active agent is an oligodynamic agent; administration of an agent that is released to dissolve organic matter that encrusts the microparticles, for instance a layered structure where one layer is comprised of active agent, a next layer comprises potassium citrate or allupurinol to dissolve “stone” material, a next layer comprises active agent, and so on; administration of a synergistic agent like urease, which prevents bacteria from changing urine pH, to ensure that the active agent is in its effective pH range; and administration of an agent that is released which facilitates microparticle dissolution, e.g. lipase, so as to promote active agent release.

Pharmacologically active agents for treating kidney stones may also be therapeutically administered according to the invention. For instance, intravesicular delivery of allopurinol using the present formulation and method facilitates the break-up of uric acid based stones and prevents the recurrence of stones, as does intravesicular administration of alkalinization agents such as acetazolamide, sodium bicarbonate, potassium citrate, and magnesium citrate, or thiazide and thiazide-like diuretics such as chlorthalidone or indapamide.

In a variation on the embodiment wherein a pharmacologically active agent is administered via the intravesicular route, a diagnostic agent is administered in the same manner, in a microparticle formulation as provided herein. The diagnostic agent is one that can be identified, quantified, or monitored using conventional imaging equipment. Diagnostic agents may be iodine-based contrast agents (e.g., iopromide, iohexol, iothalamate, ioxaglate, iopramidol, iosimenol, iodixanol, lipiodol, metrizoate, and the like) as used with voiding cystourethrography, intravenous urography, X-ray computed tomography (CT), and other diagnostic techniques; lanthanide-based contrast agents (e.g., dysprosium and gadolinium chelates, particularly gadoversetamide, gadopentetate dimeglumine, gadobutrol, and the like) and superparamagnetic iron oxide contrast agents commonly used in magnetic resonance imaging (MRI); silver-containing and gold-containing contrast agents such as PEGylated (polyethylene glycol-functionalized) silver and gold nanoparticles, proposed for use in CT imaging; microbubble-type contrast agents as used in ultrasonic imaging; and others, as will be appreciated by those of ordinary skill in the art.

### B. Controlled Release Carrier:

The controlled release microparticles herein are comprised of the pharmacologically active agent in combination with a controlled release carrier. The carrier may be in the form of a matrix in which the active agent is dispersed or embedded. Alternatively, the microparticle may be of the coated core type, wherein the controlled release carrier is in a coating on an active agent-containing core, or wherein the active agent is in a coating on a core of controlled release carrier. Generally, matrix-type microparticles are preferred, as are carrier-coated active agent-containing cores.

The choice of controlled release carrier depends on multiple factors. The carrier should be selected such that the specific gravity of a microparticle formulated with a particular active agent and the carrier is lower than that of urine. As the specific gravity of urine is in the range of about 1.005 to 1.03, the specific gravity of a microparticle formulated with the selected active agent should be less than 1.03, or less than 1.005. The carrier should also provide the desired type of controlled release, i.e., it should be gradually soluble in urine, bioerodible (e.g., by enzymatic activity), physically degradable, or some combination thereof. Hydrophilicity and hydrophobicity are also considerations. Generally, hydrophilic carriers are used with hydrophilic active agents, and hydrophobic carriers with hydrophobic active agents. The controlled release carriers are solid or semi-solid, although viscous liquid carriers can be used providing that suitable microparticles can be provided therewith in a selected liquid vehicle.

Representative carrier materials suitable for forming the microparticles herein include, but are not limited to, the following:
(1) Saturated C₆-C₃₀ fatty acids such as caproic acid, enanthic acid, caprylic acid, capric acid, caproic acid, pelargonic acid, undecylic acid, lauric acid, myristic acid, palmitic acid, stearic acid, arachidic acid, behenic acid, cerotic acid, montanic acid, and nonacosylic acid, as well as salts thereof, e.g., sodium caprylate, sodium laurate, sodium myristate, and the like;
(2) Unsaturated C₆-C₃₀ fatty acids such as crotonic acid, myristoleic acid, palmitoleic acid, oleic acid, eicosenoic acid, linoleic acid, docosadienoic acid, eicosadienoic acid, and linolenic acid;
(3) Saturated or unsaturated C₁₀-C₃₀ fatty alcohols, typically monohydric fatty alcohols, such as undecanol, lauryl alcohol, myristyl alcohol, cetyl alcohol, palmitoleyl alcohol, stearyl alcohol, oleyl alcohol, nonadecyl alcohol, behenyl alcohol, cetyl alcohol, and myricyl alcohol;
(4) Fatty acid esters, i.e., typically derived from a monohydric, dihydric, or polyhydric alcohol and at least one C₈-C₃₀ fatty acid, including:
   (a) lower alcohol fatty acid esters, i.e., esters of lower (C₂-C₆) alcohols and fatty acids, such as ethyl oleate, isopropyl myristate, and isopropyl palmitate;
   (b) triglycerides such as glyceryl tributyrate, glyceryl tricaproate, glyceryl tricaprylate, glyceryl tricaprate, glyceryl triundecanoate, glyceryl trilaurate, glyceryl trimyristate, glyceryl tripalmitate, glyceryl tristearate, glyceryl trimyristoleate, glyceryl trioleate, and mixtures thereof such as glyceryl tricaprylate/caprate/laurate, glyceryl tricaprylate/caprate/linoleate, glyceryl tricaprylate/laurate stearate, and the like;
   (c) mono- and diglycerides such as glyceryl ricinoleate, glyceryl laurate, glyceryl dilaurate, glyceryl monooleate, glyceryl dioleate, glyceryl monostearate, glyceryl behenate (e.g., Compritol®, Gattefosse), glyceryl distearate, glyceryl monopalmitate, glyceryl palmitostearate (e.g., Precirol®, Gattefosse), glyceryl caprylate/caprate (Capmul®, Abitec), caprylic acid mono and diglycerides (e.g., Imwitor 998), and mono- and di-acetylated monoglycerides (e.g., Myvacet 9-45);
   (d) polyglycerized fatty acids, i.e., polyglycerol esters of fatty acids such as polyglyceryl-2 stearate, polylglyceryl-2 oleate, polyglyceryl-2 isostearate, polyglyceryl-6-oleate, polyglyceryl-10 laurate, polyglyceryl-10 laurate, polyglyceryl-10 stearate, polyglyceryl-6 dioleate, polyglyceryl-10 trioleate, polyglyceryl polyricinoleates, tetraglyceryl pentastearate, and tetraglyceryl monostearate;
   (e) propylene glycol fatty acid esters, i.e., esters of propylene glycol and fatty acids, such as propylene glycol monolaurate, propylene glycol ricinoleate, propylene glycol monooleate, propylene glycol dioctanoate, propylene glycol stearate, and propylene glycol myristate;
   (f) polyethoxylated fatty acids, i.e., polyethylene glycol (PEG) fatty acid esters such as PEG-1 stearate, PEG-2 stearate, PEG-2 oleate, PEG-4 laurate, PEG-4 oleate, PEG-4 stearate, PEG-6 laurate, PEG-6-oleate, PEG-6 stearate, PEG-8 laurate, PEG-8 oleate, PEG-8 stearate, PEG-10 laurate, PEG-10 oleate, PEG-10 stearate, PEG-12 laurate, PEG-12 oleate, PEG-12 ricinoleate, PEG-200 oleate, PEG-400 oleate, PEG-4 dilaurate, PEG-4 dioleate, PEG-4 distearate, PEG-10 dipalmitate, PEG-8 dilaurate, PEG-8 dioleate, PEG-8 distearate, PEG-10 dipalmitate, PEG-12 dilaurate, PEG-20 distearate, PEG-32 distearate, and PEG-400 distearate;
   (g) polyethoxylated glycerol fatty acid esters, i.e., compounds wherein two of the three glycerol hydroxyl groups are esterified with a fatty acid and the third bears a PEG substituent, such as PEG-20 glyceryl laurate, PEG-30 glyceryl laurate, PEG-40 glyceryl laurate, PEG-20 glyceryl stearate, PEG-20 glyceryl oleate, and PEG-30 glyceryl oleate; and
   (h) sorbitan fatty acid esters, such as sorbitan monolaurate, sorbitan monopalmitate, sorbitan monooleate, sorbitan monostearate, sorbitan tristearate, sorbitan sesquioleate, and sorbitan trioleate, and polyethoxylated sorbitan fatty acid esters, such as PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80);
   (i) diesters formed from a C₈-C₃₀ fatty acid and a C₈-C₃₀ fatty alcohol and a carboxylic acid bearing a hydroxyl group, such as 4-hydroxycinnamic acid (coumaric acid) and w-hydroxy acids such as 16-hydroxy palmitic acid, 18-hydroxy stearic acid, and the like;
(5) phospholipids such as phosphatidylcholine, phosphatidylethanolamine, phostatidylinositol, phosphatidylserine, phosphatidyl glycerol, phosphorylated diacyl glycerides, particularly phospholipids selected from diacyl phosphatidylcholines, diacyl phosphatidylethanolamines, diacyl phosphatidylserines, diacyl phosphatidylinositols, diacyl phosphatidylglycerols, diacyl phosphatidic acids, and mixtures thereof, wherein each acyl group contains about 10 to about 30 carbon atoms and is saturated or unsaturated, and phospholipid mixtures such as lecithin, hydroxylated lecithin, and lysolesithin;
(6) sterols (including sterol derivatives) such as cholesterol, sitosterol, lanosterol, PEG-24 cholesterol ether, PEG-30 cholestanol, and phytosterol;
(7) polyethylene glycol alkyl ethers, i.e., ethers of PEG and aliphatic alcohols, such as PEG-3 oleyl ether and PEG-4 lauryl ether;
(8) polyoxyethylene-polyoxypropylene (POE-POP) block copolymers, i.e., hydrophilic surfactants with varying POE-POP ratios, typically referred to as poloxamers, and including poloxamer 105, Po108, poloxamer 122, poloxamer 123, poloxamer 124, poloxamer 181, poloxamer 182, poloxamer 183, poloxamer 184, poloxamer 185, poloxamer 188, poloxamer 212, Pod 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, as well as combinations of the foregoing and combinations with other compounds, e.g., poloxamer 407 (45%) and ammonium bicarbonate (6%). The latter combination is advantageous insofar as ammonium bicarbonate decomposes at elevated temperatures to give carbon dioxide bubbles, which in turn facilitate flotation of the microparticle formulation in the bladder.
(9) gradually erodible or degradable synthetic polymers such as polylactides, polyglycolides, polycaprolactones, polyanhydrides, polyurethanes, polyesteramides, polyorthoesters, polyvinylpyrrolidone, polyhydroxycellulose, and copolymers, derivatives, and mixtures thereof;
(10) hydrophilic polymers typically used to prepare hydrogels, e.g., cellulosic polymers such as hydroxypropyl cellulose, hydroxyethyl cellulose, hydroxypropyl methyl cellulose, methyl cellulose, ethyl cellulose, cellulose acetate, and sodium carboxymethylcellulose (NaCMC); acrylic acid polymers such as methacrylic acid, acrylic acid alkyl esters, methacrylic acid alkyl esters, and the like; copolymers of acrylic acid, methacrylic acid, acrylic acid alkyl esters, methacrylic acid alkyl esters, and the like, such as copolymers of acrylic acid, methacrylic acid, methyl acrylate, ethyl acrylate, methyl methacrylate, ethyl methacrylate, and/or hydroxyethyl methacrylate; vinyl polymers and copolymers such as polyvinyl alcohol, polyvinyl pyrrolidone, polyvinyl acetate, and ethylene-vinyl acetate copolymer; and chitosan;
(11) bile salts such as sodium cholate, sodium deoxycholate, and the like; and
(12) surfactants not encompassed by the above groups, such as sodium lauryl sulfate, cetyltrimethyl-ammonium bromide, benzalkonium chloride, 2-phenoxyethanol, and benzoyl alcohol.

It will be appreciated that the foregoing examples of controlled release carriers that can be used in the present microparticles include hydrophilic materials, hydrophobic materials, surfactants, naturally occurring materials, synthetically modified naturally occurring materials, synthetic materials; and materials having molecular weights within a relatively wide range.

The ratio of the active agent to the controlled release carrier in the formulation, as will be appreciated by those in the field of drug delivery, depends on the active agent and the intended dose to be administered, which in turn depends on the drug delivery time period. In general, the weight ratio of active agent to controlled release carrier is selected to provide drug loading (weight percent of active in the microparticles) in the range of 10% to 50% (corresponding to a weight ratio range of 1:10 to 1:1), usually in the range of 20% to 50% (corresponding to a weight ratio range of 1:5 to 1:1), and most typically in the range of 25% to 40% (corresponding to a weight ratio range of 1:3 to 2:3).

In general, the microparticles completely dissolve, degrade, or erode within the bladder, so removal of the formulation is unnecessary. In some cases, it might be desirable to facilitate microparticle degradation or removal prior to the end of the intended drug delivery period. The microparticle formulation can be removed by suction or by introduction of a microparticle degrading agent into the bladder. The degrading agent can be an enzyme, a chemical reagent such as hydrogen peroxide, or a urine acidifier such as methenamine hippurate.

In some embodiments, the microparticles are coated with or contain one or more enzymes or other biofilm disrupting agents, selected so as to degrade the extracellular matrix of a biofilm, e.g., dispersin B, trypsin (or other digestive enzymes), deoxyribonuclease, nitric oxide, etc. A microparticle coating or component can also include materials that decrease the affinity of crystals present in the urine from attaching to the microparticle surface (e.g., polyurethane or polytetrafluoroethylene) or that using an electrostatically charged material to adhere to the biomaterials (e.g., acrylamidotaurate) and cause destabilization. Any of these mechanisms for biofilm disruption and prevention of further biofilm growth can be used in combination, and some may work synergistically.

### C. Microparticle Fabrication:

Controlled release matrix microparticles: The microparticles herein having a pharmacologically active agent dispersed within a matrix of a controlled release carrier may be fabricated as follows. The selected controlled release carrier is first melted, and the active agent is then added to the melted carrier to provide an active agent-carrier admixture. The admixture is homogenized using any suitable equipment that facilitates substantially uniform distribution of the active agent in the carrier, and heating may be carried out simultaneously to ensure that the admixture remains in the form of a melt. Following at least several minutes of homogenization, the homogenized, melted admixture is fed into an atomizer so as to generate droplets of the admixture, which congeal in flight to provide the desired microparticles, which are then collected. Rotary atomizers are typically preferred. It will be appreciated that the microparticle size can be varied by controlling the feed material viscosity (for instance by adjusting the temperature of the melt), the rotational speed of the atomizer, the feed rate, and the size of the rotary disk. A representative protocol is described in Example 3.

Microparticles of the coated core type, wherein the controlled release carrier is provided as a coating on an active agent-containing core, or wherein the active agent is in a coating provided on a core comprised of the controlled release carrier, may be fabricated using conventional methods known to those of ordinary skill in the art and/or described in the pertinent texts and literature. See, for example, the description of encapsulated sustained release delivery systems in Remington: The Science and Practice of Pharmacy, 19^{th} Ed., Vol. 2 (Mack Publishing Co., 1995), at Ch. 34.

The microparticles may be fabricated so as to be at least somewhat porous, using techniques known in the art. Gas bubbles or air bubbles may be introduced into the matrix or coating, if present. Alternatively, or in addition, a porogen (e.g., a poloxamer) can be incorporated into a controlled release coating, or, for controlled release matrix-type particles, into the melt prior to, during, or after the microparticles are dispersed therein. See Cai et al. (2013), “Porous microsphere and its applications, Int J Nanomedicine 8: 1111-1120, for additional information on the preparation of porous microspheres, incorporated herein by reference.

### D. Microparticle Properties:

Although it would be conventionally assumed that controlled release microparticles are substantially spherical in shape, this is not necessarily the case. The microparticles of the invention may be spherical or substantially spherical, but the invention is not limited in this regard, insofar as the microparticles may have any three-dimensional structure that results from the selected fabrication techniques. The microparticles may be cylindrical, spherocylindrical, oval, ovoid, or the like.

The microparticles are administered as a controlled release pharmaceutical formulation for intravesicular administration to a subject, such that the formulation comprises a population of microparticles. Substantially spherical microparticles in the formulation typically have a mean diameter in the range of 500 nm to 2000 μm, such as 90 μm to 900 μm or 90 μm to 300 μm; for nonspherical microparticles, the analogous measurement is the length of the longest dimension of the microparticle (and “diameter” is used generically herein to encompass that measurement). As noted in Section 1 of this Detailed Description, the microparticle size distribution in the population of microparticles in the formulation administered to the subject is relatively narrow, i.e., the microparticles should be within 20%, preferably within 10%, of the median microparticle diameter. For example, if the median diameter is 500 μm, the particle size range should be between 400 μm and 600 μm (within 20% of the median diameter), preferably between 450 μm and 550 μm (within 10% of the median diameter. As human rugae have a cleft width in the range of 200 μm to 400 μm, a particle size of less than 400 μm is typically optimal to promote retention of microparticles within the folds of the rugae as the bladder contracts upon emptying. There will, of course, be a reduction in particle size during the drug delivery period.

A significant feature of the microparticles is their buoyancy. In one embodiment, all or substantially all of the microparticles in the microparticle population are buoyant in urine. This allows the microparticles to float to the surface of urine in the bladder, significantly reducing the fraction of the microparticle formulation that will be released with each emptying of the bladder. Buoyancy in urine is achieved by the fabrication of microparticles having a specific gravity that is generally less than 1.03, more typically less than 1.005, insofar as the specific gravity of urine is in the range of about 1.005 to 1.03.

The size and buoyancy of the microparticles provide benefits specific to the bladder. For example, in certain embodiments, the size of the microparticles allows for passage through the urethra, when needed, to prevent blockage. The buoyancy and relatively small size of the microparticles aid in retention of the microparticles in the bladder regardless of any forces acting on the particle by the bladder walls during voiding of the bladder. Microparticles that are too large can be expelled by such forces during voiding. The retention of the microparticles also benefits patients since the microparticles fill any post-void residual space of the contracted bladder, which displaces any urine that would otherwise remain within the bladder and possibly exacerbate an infection, e.g., by facilitating bacterial or fungal growth. If the contracted bladder contains excess microparticles, the size of the microparticles allows them to pass through the urethra to avoid any increase in the frequency of urination. Finally, as explained earlier herein, the contact between the microparticles and the interior walls of the bladder or urethra, as well as microparticle movement within the bladder causing turbulence in urine, substantially prevents biofilm formation and facilitates mechanical disruption of any biofilm already present, in turn reducing the likelihood of adherence of bacterial cells to a biofilm matrix.

### 3. The Intravesicular Formulation

The formulation administered to a subject to treat a urinary system disorder comprises the population of controlled release microparticles described in the preceding section, and generally includes at least one pharmaceutically acceptable excipient, i.e., a formulation component without pharmacological activity that imparts a desired physical or chemical property to the formulation or to a component thereof.

### A. Excipients:

The population of microparticles may be incorporated into a sterile liquid vehicle, which may be aqueous or nonaqueous, such that the formulation comprises an aqueous or nonaqueous dispersion, suspension, or emulsion of the microparticles in the liquid vehicle. Examples of nonaqueous liquid vehicles include fatty oils, which, it will be appreciated, comprise mixtures of fatty acids, fatty acid diglycerides, and/or fatty acid triglycerides, such as castor oil, cottonseed oil, corn oil, linseed oil, mineral oil, olive oil, sesame oil, soybean oil, and the like; fatty acids that are liquid at room temperature, i.e., lower molecular weight and/or unsaturated fatty acids, e.g., oleic acid, linoleic acid, and linolenic acid; alcohols such as such as propylene glycol, glycerol, and lower molecular weight polyethylene glycol (molecular weight less than about 750 g/mol). Ideally, the liquid vehicle should have a viscosity in the range of 2 cP to 400 cP.

The formulation may also contain, in addition to the microparticles and any liquid vehicle containing the microparticles, excipients such as buffers and other pH-adjusting agents, viscosity adjusting agents, dispersants, tonicity adjusting agents, enzyme inhibitors, preservatives and stabilizers, solubilizers, and emulsifiers.

A pH adjusting agent should maintain the pH of the formulation in the range of 5.8 to 7.4 so as not to affect the local pH of urine. Representative pH adjusting agents that serve this purpose include phosphate buffered saline (PBS) and other phosphate buffers, such as monobasic potassium phosphate, dibasic potassium phosphate, and pyrophosphate buffers; bicarbonates such as sodium bicarbonate; histidine/histidine hydrochloride; citrates such as disodium citrate and trisodium citrate; acetates such as ammonium acetate; tris(hydroxymethyl)aminomethane (Tris) buffers, arginine; and meglumine. The pH adjusting agent additionally serves to prevent crystallization of any solute on the microparticles.

Viscosity adjusting agents are thinners or thickeners, although in the present formulation any viscosity-adjusting agent is typically a thickener. Viscosity-adjusting agents herein facilitate transport of the formulation through the selected intravesicular delivery system, e.g., a catheter, intraurethral syringe, etc., and are selected to maintain a formulation viscosity in the range of 2 cP to 400 cP. Suitable viscosity adjusting agents include sodium carboxymethyl cellulose (NaCMC), sorbitol, dextran, acacia, gelatin, methylcellulose, and poly(vinylpyrrolidone). As an example, for NaCMC to maintain formulation viscosity in the aforementioned range, this generally means using a CMC with a molecular weight in the range of 90 kDa to 700 kDa, at a concentration in the range of 5 to 30 mg/mL, e.g., 5 mg/mL, 10 mg/mL, 20 mg/mL, or 30 mg/mL. Molecular weight and concentration are, of course, to be taken into account with any viscosity adjusting agent, and these parameters can be selected (molecular weight) or varied (concentration) as necessary to achieve the target viscosity. A higher molecular weight viscosity adjusting agent will result in a more viscous solution, as will a higher concentration (e.g., 5 mg/ml of 250 kDa CMC provides a viscosity similar to that obtained with 20 mg/mL of 90 kDa CMC).

Dispersants: The purpose of the dispersant is to prevent clumping and sticking of the microparticles and facilitate dispersion of the microparticles in the formulation. Dispersants, as is well known in the art, are typically, although not necessarily, surfactants; exemplary surfactants herein include the poloxamers identified in Section 2.B.8, above, and particularly Tween 20 and Tween 80, incorporated into the formulation in an amount ranging from 0.1 mg/ml to 5 mg/mL, e.g., 0.1 mg/mL, 0.5 mg/mL, 1.0 mg/mL, and 5 mg/mL (0.01%, 0.5%, 0.1%, and 0.5%, respectively). Another dispersant of interest herein is poly(vinylpyrrolidone) (PVP), e.g., Povidone K12 and Povidone K17.

Tonicity adjusting agents are incorporated to render the formulation isotonic in urine. Commonly used tonicity adjusting agents may be used herein, and include dextrose, glycerol, D-mannitol, and sodium chloride. Exemplary tonicity adjusting agents that can be incorporated into the present formulation are D-mannitol, sodium chloride, and combinations thereof; an aqueous solution with 5% D-mannitol and 0.9% sodium chloride renders the solution isotonic in urine.

Another excipient group comprises enzyme inhibitors, including inhibitors of enzymes that might degrade the pharmacologically active agent, inhibitors of enzymes present within the bladder or other region of the urinary system at a pathological level; and urease inhibitors. Insofar as urease is a known virulence factor for some urinary tract pathogens (including S. saprophyticus and P. mirabilis), incorporation of a urease inhibitor into the microparticle formulation can limit bacterial growth. Furthermore, bacterial urease alkalizes urine, causing supersaturation of calcium phosphate and struvite, consequent crystal formation, and potentially generation of a urinary stone.

Representative preservatives include antioxidants, antimicrobial agents, and chelating agents. An antioxidant preservative is useful in minimizing oxidation of the pharmacologically active agent or any excipient over the shelf life of the formulation. Commonly used antioxidant preservatives that may be advantageously used herein include ascorbic acid, ascorbyl palmitate, sodium ascorbate, acetylcysteine, monothioglycerol, and sulfurous acid salts (bisulfites, metabisulfites, and the like). Antimicrobial preservatives for preventing growth of micro-organisms in the formulation during storage and prior to use include agents such as benzalkonium chloride, benzyl alcohol, methyl paraben, propyl paraben, and thimerosal. Chelating agents such as ethylenediaminetetraacetic acid (EDTA) and salts thereof (disodium EDTA, tetrasodium EDTA, sodium calcium edetate, etc.) are useful to sequester metal ions in the formulation that could otherwise facilitate unwanted enzymatic or other reactions.

If the formulation is an emulsion, as may be the case with hydrophobic microparticles in a hydrophilic vehicle, hydrophilic microparticles in a hydrophobic vehicle, a mixture of hydrophilic and hydrophobic microparticles, or a mixture of hydrophilic and hydrophobic excipients, use of an emulsifier is recommended to facilitate homogeneous dispersion of microparticles and/or excipients in a liquid vehicle. Emulsifiers, as known in the art, are surfactants, comprised of a polar or charged hydrophilic moiety and a non-polar lipophilic (hydrophobic) moiety. Emulsifiers herein can also serve as a dispersant, stabilizer, and/or the liquid vehicle. Suitable emulsifiers for incorporation into the present formulation include any emulsifiers that are typically used in non-solid pharmaceutical preparations, such as poloxamers (including those identified in Section 2.B.8, above), polyoxyethylene ethers, polyethoxylated castor oil, polyoxyethylene fatty acid esters (polysorbates), polyoxyethylene stearates, propylene glycol alginate, sodium citrate, sorbitan fatty acid esters, lecithin, and diethanolamine. See, e.g., Handbook of Pharmaceutical Excipients, 3^{rd} Edition, Kibbe, ed. (American Pharmaceutical Association, 2000).

### B. Microparticle Combinations:

The intravesicular formulation can comprise two or more populations of microparticles, with each population differing from each other population in at least one respect. In another embodiment, then, the formulation additionally comprises a secondary population of microparticles that differs from the primary population of microparticles, for example, comprising a different pharmacologically active agent, a different amount of a pharmacologically active agent, a different controlled release carrier, or a different amount of controlled release carrier, or having a different controlled release profile or specific gravity.

For example, a first, or primary, population of microparticles may provide a first drug release profile (e.g., substantially immediate release of a bolus dose) and second, or secondary, population of microparticles may provide a second drug release profile different from the first (e.g., a sustained release profile). As another example, one population of microparticles can contain a first pharmacologically active agent and a second population of microparticles can contain a second pharmacologically active agent, and the two populations may or may not be formulated so as to have different drug release profiles. In some cases, it may be desirable that some fraction of the microparticles administered to the bladder sink to the trigone and exhibit the intended pharmacological activity there—for instance to disrupt a biofilm at the trigone, to deliver an anesthetic agent to the trigone, or to administer an anti-infective agent to the trigone—an additional population of microparticles can be fabricated so as to be less buoyant than the first population. As alluded to earlier herein, this can be achieved by selecting a different controlled release carrier, a different carrier-to-active agent ratio, or the like. Inclusion of less buoyant particles in the formulation can assist in delivery of an active agent to regions of the bladder that retain urine after contraction, for instance a cystocele (also known as a prolapsed, herniated, dropped, or fallen bladder). With a cystocele, the ligaments that hold the bladder and surrounding muscles stretch or weaken, allowing the bladder to sag, and it is the sagging region that can be treated by deposition of the less buoyant or nonbuoyant particles in that area.

### C. Representative Formulations and Dosage Considerations:

Representative microparticle formulations according to the invention are as follows:
15 wt. % SSD, 85 wt. % Dynasan 118;
25 wt. % SSD, 75 wt. % Dynasan 118;
40 wt. % SSD, 60 wt. % Dynasan 118;
15 wt. % SSD, 85 wt. % glyceryl tripalmitate;
25 wt. % SSD, 75 wt. % glyceryl tripalmitate;
40 wt. % SSD, 60 wt. % glyceryl tripalmitate;
15 wt. % SSD, 85 wt. % glyceryl behenate;
25 wt. % SSD, 75 wt. % glyceryl behenate;
40 wt. % SSD, 60 wt. % glyceryl behenate;
15 wt. % SSD, 85 wt. % stearyl alcohol;
25 wt. % SSD, 75 wt. % stearyl alcohol;
40 wt. % SSD, 60 wt. % stearyl alcohol;
15 wt. % SSD, 85 wt. % cetyl alcohol;
25 wt. % SSD, 75 wt. % cetyl alcohol;
15 wt. % silver nitrate, 85 wt. % Dynasan 118;
25 wt. % silver nitrate, 75 wt. % Dynasan 118;
40 wt. % silver nitrate, 60 wt. % Dynasan 118;
15 wt. % silver nitrate, 85 wt. % glyceryl tripalmitate;
25 wt. % silver nitrate, 75 wt. % glyceryl tripalmitate;
40 wt. % silver nitrate, 60 wt. % glyceryl tripalmitate;
15 wt. % silver nitrate, 85 wt. % glyceryl behenate;
25 wt. % silver nitrate, 75 wt. % glyceryl behenate;
40 wt. % silver nitrate, 60 wt. % glyceryl behenate;
15 wt. % silver nitrate, 85 wt. % stearyl alcohol;
25 wt. % silver nitrate, 75 wt. % stearyl alcohol;
40 wt. % silver nitrate, 60 wt. % stearyl alcohol;
15 wt. % silver nitrate, 85 wt. % cetyl alcohol;
25 wt. % silver nitrate, 75 wt. % cetyl alcohol;
15 wt. % fosfomycin, 85 wt. % Dynasan 118;
25 wt. % fosfomycin, 75 wt. % Dynasan 118;
40 wt. % fosfomycin, 60 wt. % Dynasan 118;
15 wt. % fosfomycin, 85 wt. % glyceryl tripalmitate;
25 wt. % fosfomycin, 75 wt. % glyceryl tripalmitate;
40 wt. % fosfomycin, 60 wt. % glyceryl tripalmitate;
15 wt. % fosfomycin, 85 wt. % glyceryl behenate;
25 wt. % fosfomycin, 75 wt. % glyceryl behenate;
40 wt. % fosfomycin, 60 wt. % glyceryl behenate;
15 wt. % fosfomycin, 85 wt. % stearyl alcohol;
25 wt. % fosfomycin, 75 wt. % stearyl alcohol;
40 wt. % fosfomycin, 60 wt. % stearyl alcohol;
15 wt. % fosfomycin, 85 wt. % cetyl alcohol;
25 wt. % fosfomycin, 75 wt. % cetyl alcohol;
40 wt. % fosfomycin, 60 wt. % cetyl alcohol;
40 wt. % fosfomycin, 60 wt. % cetyl alcohol;
15 wt. % lidocaine, 85 wt. % Dynasan 118;
25 wt. % lidocaine, 75 wt. % Dynasan 118;
40 wt. % lidocaine, 60 wt. % Dynasan 118;
15 wt. % lidocaine, 85 wt. % glyceryl tripalmitate;
25 wt. % lidocaine, 75 wt. % glyceryl tripalmitate;
40 wt. % lidocaine, 60 wt. % glyceryl tripalmitate;
15 wt. % lidocaine, 85 wt. % glyceryl behenate;
25 wt. % lidocaine, 75 wt. % glyceryl behenate;
40 wt. % lidocaine, 60 wt. % glyceryl behenate;
15 wt. % lidocaine, 85 wt. % stearyl alcohol;
25 wt. % lidocaine, 75 wt. % stearyl alcohol;
40 wt. % lidocaine, 60 wt. % stearyl alcohol;
15 wt. % lidocaine, 85 wt. % cetyl alcohol;
25 wt. % lidocaine, 75 wt. % cetyl alcohol;
40 wt. % lidocaine, 60 wt. % cetyl alcohol;
15 wt. % mitomycin, 85 wt. % Dynasan 118;
25 wt. % mitomycin, 75 wt. % Dynasan 118;
40 wt. % mitomycin, 60 wt. % Dynasan 118;
15 wt. % mitomycin, 85 wt. % glyceryl tripalmitate;
25 wt. % mitomycin, 75 wt. % glyceryl tripalmitate;
40 wt. % mitomycin, 60 wt. % glyceryl tripalmitate;
15 wt. % mitomycin, 85 wt. % glyceryl behenate;
25 wt. % mitomycin, 75 wt. % glyceryl behenate;
40 wt. % mitomycin, 60 wt. % glyceryl behenate;
15 wt. % mitomycin, 85 wt. % stearyl alcohol;
25 wt. % mitomycin, 75 wt. % stearyl alcohol;
40 wt. % mitomycin, 60 wt. % stearyl alcohol;
15 wt. % mitomycin, 85 wt. % cetyl alcohol;
25 wt. % mitomycin, 75 wt. % cetyl alcohol;
40 wt. % mitomycin, 60 wt. % cetyl alcohol;
15 wt. % gemcitabine, 85 wt. % Dynasan 118;
25 wt. % gemcitabine, 75 wt. % Dynasan 118;
40 wt. % gemcitabine, 60 wt. % Dynasan 118;
15 wt. % gemcitabine, 85 wt. % glyceryl tripalmitate;
25 wt. % gemcitabine, 75 wt. % glyceryl tripalmitate;
40 wt. % gemcitabine, 60 wt. % glyceryl tripalmitate;
15 wt. % gemcitabine, 85 wt. % glyceryl behenate;
25 wt. % gemcitabine, 75 wt. % glyceryl behenate;
40 wt. % gemcitabine, 60 wt. % glyceryl behenate;
15 wt. % gemcitabine, 85 wt. % stearyl alcohol;
25 wt. % gemcitabine, 75 wt. % stearyl alcohol;
40 wt. % gemcitabine, 60 wt. % stearyl alcohol;
15 wt. % gemcitabine, 85 wt. % cetyl alcohol;
25 wt. % gemcitabine, 75 wt. % cetyl alcohol; and
40 wt. % gemcitabine, 60 wt. % cetyl alcohol.

It will be appreciated from the discussion in Section 3.A and elsewhere in this application that the intravesicular formulation can comprise additional components, including additional microparticle types (e.g., with different active agents, loading %, and/or different controlled release carriers), a liquid carrier in which the microparticles are dispersed, and excipients such as pH-adjusting agents, viscosity adjusting agents, dispersants, tonicity adjusting agents, preservatives and stabilizers, solubilizers, and emulsifiers.

The target concentration of pharmacologically active agent in the bladder is generally in the range of 10 ppm to 150 ppm, i.e., 10 mg/L to 150 mg/L, in urine, e.g., 10 ppm, 16 ppm, 20 ppm, 35 ppm, 45 ppm, 50 ppm, 75 ppm, 100 ppm, 115 ppm, 125 ppm, and 128 ppm (corresponding to 10 mg/L, 16 mg/L, 20 mg/L, 35 mg/L, 45 mg/L, 50 mg/L, 75 mg/L, 100 mg/L, 115 mg/L, 125 mg/L, and 128 mg/L, respectively), etc., but will, of course, depend on multiple factors, including the active agent administered, the indication, the age, weight, and condition of the subject, and the like. A preferred sub-range is 15 ppm to 130 ppm. Dosage may be calculated by extrapolating from the following example. For a drug delivery period of 90 days, about 100 L will pass through the bladder. Assuming that 100% of the active agent in the microparticles will be released into the bladder during the 90-day period, achieving a 20 mg/L concentration requires that 2000 mg (2 g) active agent, or 2 g, be delivered to treat the 100 L of urine. For a population of microparticles that comprises 25% active agent and 75% controlled release carrier, 8 g of microparticles would have to be administered in order to deliver 1 g of the active agent into the bladder. As another example, during a drug delivery period of 180 days, about 200 L of urine will pass through the bladder. Again, assuming that 100% of the active agent in the microparticles is released into the bladder during the 180-day period, achieving a 50 mg/L concentration requires that 10,000 mg (10 g) be delivered to treat the 200 L of urine. For a population of microparticles that, for purposes of illustration, again comprises 25 wt. % active agent and 75 wt. % controlled release carrier, 40 g of microparticles would have to be administered to deliver 10 g of the active agent to treat the 200 L of urine. The calculation can be readily adjusted for different active agent-to-carrier ratios, drug delivery time periods, and target concentration of active agent in the bladder. It should also be noted that as urine production varies throughout the day, the concentration of active agent within the bladder varies throughout the day as well (and may peak during a time of low urine production).

The extended drug delivery time period during which the active agent is released from the microparticles into the bladder and, preferably, during which therapeutically effective concentrations of the active agent or a metabolite thereof are provided in the bladder, is in the range of two hours to one year, e.g., two hours to six months, two hours to four months, two hours to three months, two hours to one month, 48 hours to six months, 48 hours to four months, 48 hours to three months, 48 hours to one month, two weeks to four months, two weeks to three months, or two hours to one month.

### 4. Indications and Administration

The microparticle formulations of the invention can be administered to a subject to treat a disorder of the urinary system. The disorder may be a disease or other adverse condition of the bladder, kidneys, ureters, and/or urethra, and is not limited in any respect except that the disorder is responsive or predicted to be responsive to the intravesicular administration of a particular pharmacologically active agent or type of pharmacologically active agent. Examples of urinary system disorders that can be treated according to the invention include urinary system infections (commonly referred to as “UTIs” or urinary tract infections”) such as bacterial, fungal, and viral infections; cancers or benign tumors of the urinary system; urinary incontinence (including urge urinary incontinence, or overactive bladder, “OAB”) and urinary retention; urinary system inflammation, injury, or scarring; and kidney stones, diabetic nephropathy, or kidney failure.

Examples of specific indications and representative active agents to treat the indications are as follows:
cystitis (an infection of the lower urinary tract), including acute cystitis, chronic cystitis, hemorrhagic cystitis, bacterial cystitis, and emphysematous cystitis, any of which can be caused by a bacterial, fungal, or viral infection and are treated accordingly, with an appropriate anti-infective agent (see Section 2.A for representative antibacterial, anti-fungal, and antiviral agents);
interstitial cystitis (also known as bladder pain syndrome, BPS), commonly treated with a serotonin reuptake inhibitor (SRI) such as a tricyclic antidepressant (amitriptyline, imipramine, desipramine, etc.), a serotonin-norepinephrine reuptake inhibitor (such as duloxetine or venlafaxine), an anti-inflammatory agent (as described in Section 2.A; ibuprofen is typical), or pentosan polysulfate;
neurogenic bladder dysfunction, commonly treated with an anti-cholinergic agent (e.g., oxybutynin or tolterodine) to reduce bladder contractions, or with a muscarinic agent (e.g., bethanechol or an α-blocker such as phentolamine) to treat overflow incontinence;
overactive bladder (OAB; also referred to as “urge urinary incontinence”) syndrome, commonly treated with an antimuscarinic drug (e.g., darifenacin, hyoscyamine, oxybutynin, tolterodine, solifenacin, trospium, or fesoterodine), or with a β3-adrenergic receptor agonist (such as mirabegron or vibegron);
incontinence caused by OAB, typically treated with an anesthetic agent or an analgesic agent such as those identified in Section 2.A;
stress incontinence, also known as stress urinary incontinence (SUI), resulting from inadequate closure of the bladder outlet by the urethral sphincter,
chlamydia (Chlamydia trachomatis) and gonorrhea (Neisseria gonorrhoeae), commonly treated with an antibiotic such as azithromycin, doxycycline, erythromycin, levofloxacin, ofloxacin, or tetracycline (see Section 2.A for additional representative active agents for treating chlamydia);
gonococcal urethritis, caused by an N. gonorrhoeae infection, typically treated with ceftriaxone or ceftriaxone, in combination with azithromycin;
non-gonococcal urethritis, usually caused by Chlamydia trachomatis, Trichomonas vaginalis, adenoviridae, uropathogenic E. coli, HSV1 or HSV-2, cytomegalovirus, Ureaplasma urealyticum, methicillin-resistant S. aureus, or Group B streptococcus, most commonly caused by chlamydia, is typically treated with an antibiotic such as azithromycin, doxycycline, erythromycin, levofloxacin, or ofloxacin;
syphilis, commonly treated with benzathine benzylpenicillin or ceftriaxone;
pyelonephritis, commonly treated with a fluoroquinolone antibiotic (e.g., ciprofloxacin or levofloxacin), a cephalosporin antibiotic (e.g., ceftriaxone), an aminoglycoside antibiotic (e.g., gentamycin), or fosfomycin;
kidney stones, which can be treated with, for instance, allopurinol, alkalizing agents, or thiazide diuretics, as explained in Section 2.A;
pain associated with kidney stones, typically treated with an NSAID anti-inflammatory/analgesic or with an opioid analgesic;
diabetic nephropathy (diabetic kidney disease), treated with an inhibitor of the renin-angiotensin-aldosterone system (RAAS) such as an angiotensin converting enzyme (ACE) inhibitor, an angiotensin receptor blocker, a direct renin inhibitor, or a mineralocorticoid antagonist, or with an anti-diabetic agent (see Section 2.A);
vesicoureteral reflux (VUR), a disorder that increases the likelihood of a urinary system infection, particularly in children; and
cancers or benign tumors of the urinary system, treated with a chemotherapeutic agent as set forth in Section 2.A, particularly apaziquone, fosfomycin, gemcitabine, and BCG immunotherapy.

The intravesicular formulation is administered using any known or hereinafter developed device suitable for introducing a pharmaceutical formulation into bladder. For instance, the formulation may be administered through a transurethral syringe, a Toomey syringe, or via a catheter.

Intravesicular administration of a formulation of the invention is particularly useful in the treatment of individuals for whom a therapeutically effective dose of an oral medication is likely to be problematic, including the elderly, who make up the majority of individuals afflicted with recurring and/or serious urinary system disorders, and children. The invention additionally finds utility in the treatment of individuals afflicted with any of a wide range of conditions and diseases that increase the likelihood of a urinary system disorder, particularly complicated urinary tract infections. Such individuals include subjects with lupus and other systemic autoimmune diseases; subjects on immunosuppressive therapy; subjects with a neurological disorder; subjects with a sexually transmitted disease; diabetic patients; cancer patients; patients with a functional or anatomic abnormality of the urinary tract; patients who experienced a UTI as a child; patients who have had extensive antimicrobial therapy; patients who have contracted a nosocomial infection; and numerous others.

### EXPERIMENTAL

### Example 1

### Antimicrobial Efficacy of SSD in Artificial Urine:

The antimicrobial efficacy of SSD was evaluated in artificial urine against various test microbes by determining the planktonic log reductions after specific contact times. The testing was designed to measure % kill and log reduction of planktonic cultures of several microbes after six different exposure times. The SSD was tested at four different dilutions to demonstrate the kill rate of the silver ions.

Challenge time, dilution range for silver ions, and dilution range for SSD are provided in Table 1:

TABLE 1

| | Challenge time | Dilutions (Ag⁺), | Dilutions (SSD), |
|---|---|---|---|
| Sample | (hr) | mg/L | mg/L |
| SSD | 0, 1, 2, 4, 6 | 3, 5, 10, 20 | 9.93, 16.55, |
| | | | 33.11, 66.22 |
| Negative | 0, 1, 2, 4, 6 | — | — |
| control | | | |

Artificial urine was prepared as described by Brooks et al. (1997), “A simple artificial urine for growth of urinary pathogens,” Lett Appl Microbiol 24(3): 203-6, sterilized by filtration through 0.2 μm filter units, and stored at 4° C.

Microorganisms, growth media, and conditions are set forth in Table 2:

TABLE 2

| Organism | Growth Media and Conditions* |
|---|---|
| Escherichia coli | Tryptic soy agar (TSA), humidified |
| ATCC 700928 | incubator at 37 ± 2° C. for 24 hr |
| Klebsiella pneumonia | TSA, humidified incubator at |
| ATCC BAA-1705 | 37 ± 2° C. for 24 hr |
| Staphylococcus | TSA, humidified incubator at |
| saprophyticus | 37 ± 2° C. for 24 hr |
| ATCC 15305 | |
| Proteus mirabilis | TSA, humidified incubator at |
| ATCC 29906 | 37 ± 2° C. for 24 hr |
| Enterococcus faecalis | TSA, humidified incubator at |
| ATCC 29212 | 37 ± 2° C. for 24 hr |
| Staphylococcus aureas | TSA, humidified incubator at |
| ATCC 6538 | 37 ± 2° C. for 24 hr |
| Candida albicans | TSA, Sabouraud dextrose broth and agar |
| ATCC 18804 | (SDB/SDA), humidified incubator at |
| | 37 ± 2° C. for 24 hr |
| Streptococcus agalactiae | TSA, humidified incubator at |
| GP #7 | 37 ± 2° C. for 24 hr |

The organisms in Table 2 were tested using exposure time points of 1, 2, 4, and 6 (as indicated in Table 1) from the same plates over time. Three plates were needed per strain, for a total of 27 challenge plates. Three replicate samples were used, with sterility controls and growth controls used for each experiment.

Working solutions were made up of either the test or control compositions according to Table 1, and 4.8 mL of the working solutions were added to the appropriate wells of the challenge plates.

### Inoculum Preparation:

Using a cryogenic stock (at −70° C.), the first sub-culture of the microorganisms was struck out on appropriate media (TSA plates for bacterial isolates or SDA plates for C. albicans, as indicated in Table 2).

Plates were incubated at 37±2° C. for 24 hours and stored at 4±1° C. until needed.

From the first sub-cultures, second sub-cultures were struck out on appropriate media (again, TSA for the bacterial isolates or SDB/SDA for C. albicans).

From the first sub-cultures, second sub-cultures were struck out on appropriate media (TSA for the bacterial isolates or SDB/SDA for C. albicans).

Approximately 4-5 large, or 5-10 small, well-isolated colonies from the second subculture plate were emulsified in distilled, deionized water and adjusted to achieve a turbidity equivalent to a 0.5 McFarland standard.

Bacteria were emulsified in 6 mL water, while C. albicans was emulsified in 12 ml distilled water.

Cell suspensions were centrifuged (3000×g for 10 min), and the pellets were washed 2× by decanting the supernatant and resuspending the cell pellet in equal volumes of distilled deionized water.

Following the washing, the cell pellets were resuspended in distilled deionized water Bacteria were resuspended in 20 mL water, while C. albicans was resuspended in 4 ml distilled water.

The cell density of the inoculum was confirmed by serially diluting and spot plating.

A purity check was performed by checking the spot plates for the presence of abnormal looking colonies after 16-20 hours of incubation. All strains were determined to be pure cultures.

### Inoculation of Challenge Plates:

200 μL of the inoculum was added to each well of the challenge plates, except the sterility control (SC) wells. 200 μL of sterile water was added to the SC wells. Inoculated plates were incubated at 37±2° C. in a non-CO₂ incubator and samples were taken at 0, 1, 2, 4 and 6 hr timepoints.

### Recovery:

Following each of the challenge time points, 100 μL was mixed with 100 μL modified D/E neutralizer composition (D/E neutralizing broth, 5 g/L L-cysteine, and 5 g/L glutathione, 2× strength) in the most concentrated of the serial dilution plates. 180 μL of sterile 0.9% saline was placed in the remaining dilutions. Serial dilutions of 1 to 107 were prepared. 10 μL from each well was then removed and spot plated onto prepared TSA or SDA plates. Plates were incubated at 37±2° C. and the number of resulting colonies were counted after approximately 16-24 hours of incubation. Data was evaluated as log 10 CFU/mL.

### Neutralization Validation:

200 μL of each inoculum was placed into triplicate wells, per strain, containing 2 mL of the modified D/E neutralizer composition, and 1.8 mL of the 20 ppm Ag+ test solution. After 5 min, 100 μL from each well of the neutralization efficacy plates was pipetted into the first row of a 96-well microtiter plate. 180 μL of sterile 0.9% saline was placed in the remaining rows. Serial dilutions were prepared, 10 μL from each well was removed and spot plated, plates were incubated, and data evaluated as described under “Recovery.”

### Calculations and Statistics:

Spot CFU/mL=(CFU/10 μL)/0.010

Log₁₀(CFU/mL)=Log₁₀(CFU/mL+1)

Log₁₀ reduction=Log₁₀(CFU/mL)(growth control)−Log₁₀(CFU/mL)(test conditions)

% Kill=((antilog avg(growth control)−antilog avg(test conditions))/antilog avg(growth control))×100

P-values compared to the growth control were a one-way ANOVA with a Tukey multiple comparisons Post Test.

### Results:

A Log₁₀ reduction >4 was observed for E. coli, K. pneumoniae, P. mirabilis and C. albicans at all four concentrations of SSD tested, after 4 hours. A Log₁₀ reduction >3 was observed for S. saprophyticus, after 6 hours, at all four concentrations of SSD tested. There was no detectable difference in the amount, or rate, of killing between the four different concentrations of SSD tested.

### Example 2

### Antimicrobial Efficacy of SSD and Cefpodoxime Against UTI Bacteria Isolates:

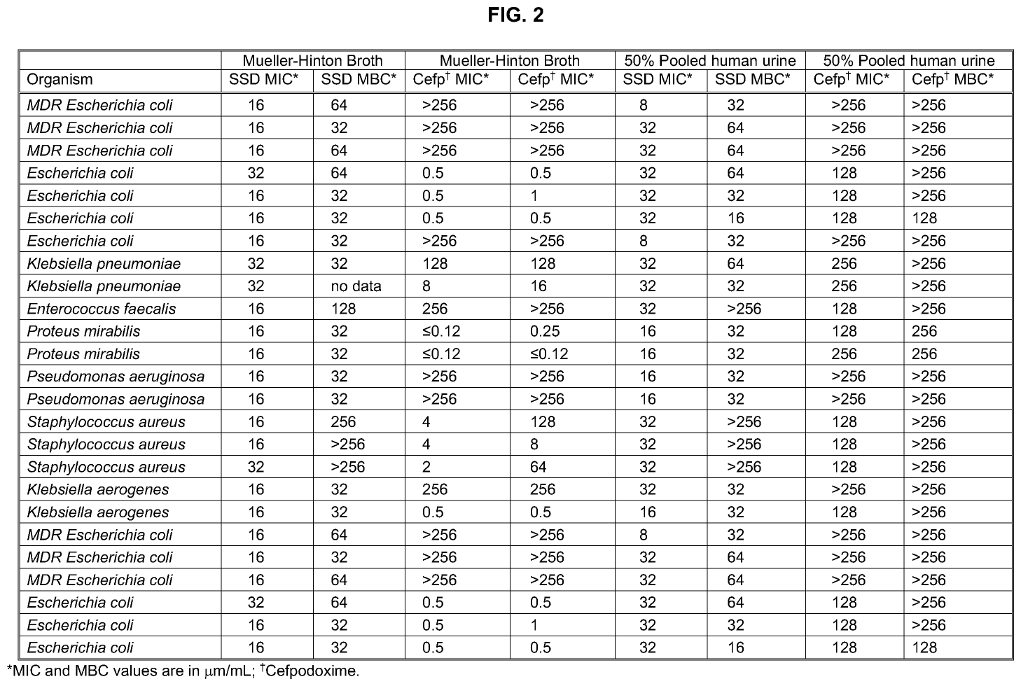

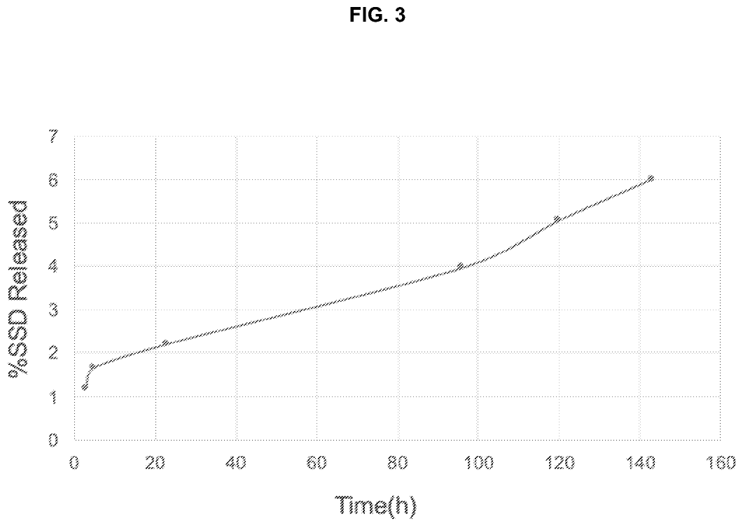

The antimicrobial efficacy of SSD and cefpodoxime was evaluated with respect to the bactericidal effect of SSD against the organisms listed in Table 2 (FIG. 3) in addition to two E. coli multi-drug resistant (MDR) clinical isolates. Cefpodoxime, a commonly prescribed antibiotic, was used as a comparator agent. The minimum inhibitory concentration (MIC) and minimum bactericidal concentration (MBC) were identified. These define a drug's potency in terms of the concentration at which it will inhibit growth of (MIC) or completely kill (MBC) 1×10⁶ challenge microorganisms during an 18 hr to 20 hr period of incubated (35±2° C.) exposure. MIC values for SSD and cefpodoxime against each of the 10 isolates were generated using reference frozen-form broth microdilution susceptibility panels and 50% pooled human urine in accordance with Clinical and Laboratory Standards Institute (CLSI) guidelines. Both SSD and cefpodoxime were tested over 15 doubling dilutions (1:2 serial dilutions) and bacterial colony counts were done on each isolate tested to determine the starting inoculum concentrations (target 5×105 CFU/mL). The results are shown in FIG. 2 (Table 3).

### Example 3

### Microparticle Fabrication:

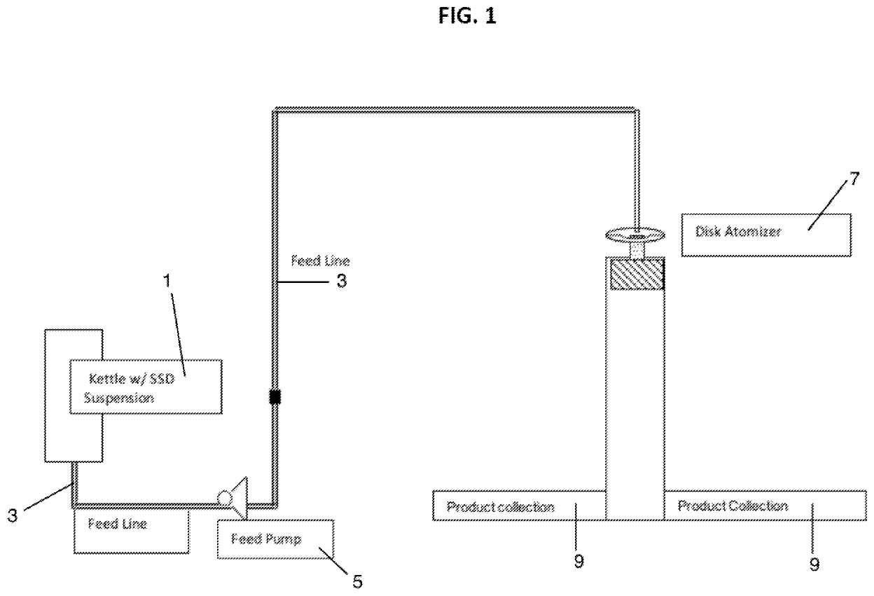

A Melt Spray Congealing (MSC) process was used to prepared microparticles with glyceryl tristearate (Dynasan® 118, from IOI Oleochemicals GmbH, hereinafter “Dynasan”) as the controlled release carrier and silver sulfadiazine (SSD), an antibiotic, as the pharmacologically active agent. The process involves dispersing SSD in a Dynasan melt and then atomizing the composition so provided using a rotary atomizer. The atomized droplets are allowed to congeal resulting in SSD being entrapped in the Dynasan matrix. The MSC fabrication process is illustrated schematically in FIG. 1. The particle size of the MSC microparticles can be adjusted by controlling the feed material viscosity, rotational speed, and feed rate, as well as disk design and disk size. Particle size is, for instance, inversely proportional to feed material viscosity, rotational speed, and feed rate.

MSC protocol used to prepare microparticles with 25% silver sulfadiazine and 75% Dynasan: Referring to FIG. 1, the Dynasan (40 g) was melted in a kettle 1 at 90° C. After melting was complete, SSD (13.3 g) was added to the kettle containing the melted Dynasan and dispersed therein using homogenization up to 20,000 rpm for approximately 3 minutes. The SSD suspension so prepared was then fed at a rate of 75 g/min to 100 g/min) into feed line 3 and through feed pump 5 onto a 4″-disk rotary atomizer 7 heated to approximately 90° C. to 100° C. and rotating at approximately 2400 rpm. The SSD suspension formed a thin film across the disk surface and was atomized into distinct droplets at its periphery. The atomized droplets congealed in flight and were collected (40 g, 75% yield) on a tray 9, but can be collected in a plastic enclosure, or by use of a cyclone separator, or using other suitable means. To ensure a narrow particle size distribution, a sieve can be used to sift out microparticles outside of the desired size range.

### Example 4

### Microparticle Density Evaluations:

Microparticles were prepared with SSD and a controlled release carrier (CRC) at different ratios, and microparticle density was evaluated. Density of various materials are provided in Table 4, while CRCs, SSD:CRC weight ratios in the microparticles, CRC wt. %, CCR density, and (theoretical) microparticle density are set forth in Table 5:

TABLE 4

| | Density |
|---|---|
| Material | (g/cm³) |
| SSD | 1.496 |
| Glyceryl trimyristate | 0.885 |
| Glyceryl tripalmitate | 0.8752 |
| Glyceryl tristearate | 0.862 |
| Stearyl alcohol | 0.812 |
| Glyceryl behenate | 0.942 |
| Poloxamer P407 | 1.05 |
| Urine | 1.027 |
| 0.9% saline | 1.0046 |

TABLE 5

| | | | | CCR | Microparticle |
|---|---|---|---|---|---|
| | | SSD:CCR | CCR | Density | density |
| # | CCR | (wt/wt) | wt. % | (g/cm³) | (g/cm³) |
| 1 | Glyceryl trimyristate | 10:90 | 90 | 0.885 | 21.0965 |
| 2 | Glyceryl trimyristate | 25:75 | 75 | 0.885 | 51.41375 |
| 3 | Glyceryl trimyristate | 40:60 | 60 | 0.885 | 81.731 |
| 4 | Glyceryl tripalmitate | 10:90 | 90 | 0.8752 | 21.08768 |
| 5 | Glyceryl tripalmitate | 25:75 | 75 | 0.8752 | 51.4064 |
| 6 | Glyceryl tripalmitate | 40:60 | 60 | 0.8752 | 81.72512 |
| 7 | Glyceryl tristearate | 10:90 | 90 | 0.862 | 21.0758 |
| 8 | Glyceryl tristearate | 25:75 | 75 | 0.862 | 51.3965 |
| 9 | Glyceryl tristearate | 40:60 | 60 | 0.862 | 81.7172 |
| 10 | Stearyl alcohol | 10:90 | 90 | 0.812 | 21.0308 |
| 11 | Stearyl alcohol | 25:75 | 75 | 0.812 | 51.359 |
| 12 | Stearyl alcohol | 40:60 | 60 | 0.812 | 81.6872 |
| 13 | Glyceryl behenate | 10:90 | 90 | 0.942 | 21.1478 |
| 14 | Glyceryl behenate | 25:75 | 75 | 0.942 | 51.4565 |
| 15 | Glyceryl behenate | 40:60 | 60 | 0.942 | 81.7652 |
| 16 | Poloxamer P407 | 10:90 | 90 | 1.05 | 21.245 |
| 17 | Poloxamer P407 | 25:75 | 75 | 1.05 | 51.5375 |
| 18 | Poloxamer P407 | 40:60 | 60 | 1.05 | 81.83 |

### Example 5

### Microparticle Release Profile:

Microparticles containing 25 wt. % SSD and 75 wt. % Dynasan® 118 were prepared as described in Example 4, and dispersed in PBS buffered to a pH of 7.4. The concentration of microparticles in PBS was 10 mg/mL, with a corresponding concentration of 2.5 mg/mL for active agent in the formulation. The mean particle diameter (i.e., d(0.5)) was found to be 174 μm.

The amount of drug released over time was evaluated using a dissolution test conducted at 37° ° C. with constant stirring. The results are presented in Table 6, which shows the amount of SSD released, the percentage of total SSD released, and the rate of SSD release (% SSD released per hr) at different time points during the evaluation:

TABLE 6

| | SSD mg | % SSD | % SSD |
|---|---|---|---|
| Time (hr) | released | released | released/hr |
| 3 | 0.027 | 1.175 | 0.392 |
| 5 | 0.038 | 1.654 | 0.631 |
| 23 | 0.051 | 2.202 | 0.662 |
| 96 | 0.092 | 3.962 | 0.686 |
| 120 | 0.117 | 5.059 | 0.732 |
| 143 | 0.139 | 6.010 | 0.773 |

The percentage of SSD released over time was plotted. The results, shown in FIG. 3, illustrate that zero order release kinetics were achieved, i.e., the SSD was released from the microparticles at an approximately constant rate. At the release rate calculated, the microparticle formulation would provide controlled release drug delivery in the bladder for up to three months.

1. A controlled release pharmaceutical formulation for intravesical administration to a subject, the formulation comprising a population of microparticles having a mean diameter of 500 nm to 2000 μm and comprised of 2.5 wt. % to 95 wt. % of a pharmacologically active agent and 5 wt. % to 97.5 wt. % of a controlled release carrier,
wherein the active agent, carrier, and relative amounts of the active agent and carrier in the microparticles render the microparticles buoyant in urine.

2. The formulation of claim 1, wherein the microparticles have a mean specific gravity of less than 1.03.

3.-4. (canceled)

5. The formulation of claim 4, wherein the carrier provides for controlled release of the pharmacologically active agent in the urinary system and/or bladder.

6.-7. (canceled)

8. The formulation of claim 1, wherein the carrier is comprised of a matrix and the pharmacologically active agent is dispersed therein.

9. The formulation of claim 1, wherein the carrier comprises a coating on a core that comprises the pharmacologically active agent dispersed therein.

10. The formulation of claim 1, wherein the carrier gradually dissolves, degrades, or erodes in urine to release the pharmacologically active agent from the microparticles.

11. The formulation of claim 1, wherein the carrier comprises a fatty acid, fatty alcohol, fatty acid ester, phospholipid, sterol, polyethylene glycol alkyl ether, polyoxyethylene-polyoxypropylene block copolymer, chitosan, or bile salt.

12. (canceled)

13. The formulation of claim 1, wherein the carrier comprises a fatty acid ester, the fatty acid ester comprised of a lower alcohol fatty acid ester, a triglyceride, a monoglyceride, a triglyceride, a polyglycerized fatty acid, a propylene glycol fatty acid ester, a polyethoxylated fatty acid, a polyethoxylated glyceryl fatty acid ester, a sorbitan fatty acid ester, or a hydroxyacid diester.

14. The formulation of claim 13, wherein the fatty acid ester is a triglyceride selected from glyceryl tributyrate, glyceryl tricaproate, glyceryl tricaprylate, glyceryl tricaprate, glyceryl triundecanoate, glyceryl trilaurate, glyceryl trimyristate, glyceryl tripalmitate, glyceryl tristearate, glyceryl trimyristoleate, and glyceryl trioleate.

15. (canceled)

16. The formulation of claim 1, wherein the carrier comprises a surfactant selected from sodium lauryl sulfate, cetyltrimethyl-ammonium bromide, benzalkonium chloride, 2-phenoxyethanol, and benzoyl alcohol.

17. The formulation of claim 1, wherein the pharmacologically active agent comprises one or more of an anti-infective agent, an anesthetic agent, an analgesic agent, a diuretic, an anti-inflammatory agent, a coagulant or an anti-coagulant, a chemotherapeutic agent, an agent for the treatment of incontinence, an angiotensin-aldosterone system (RAAS) inhibitor, an agent for treating kidney stones, and a contrast agent for diagnostics and monitoring.

18.-23. (canceled)

24. The formulation of claim 5, wherein the controlled release carrier provides sustained release of the pharmacologically active agent in the bladder over a drug delivery time period of 2 hours to six months.

25. (canceled)

26. The formulation of claim 24, wherein the controlled release carrier provides for approximately zero order release of the pharmacologically active agent in the bladder.

27. The formulation of claim 1, further including a liquid vehicle in which the population of microparticles is dispersed, the liquid vehicle comprised of a viscosity adjusting agent, a tonicity adjusting agent, a buffer, and a dispersant.

28.-34. (canceled)

35. A method of treating a urinary system disorder in a subject, the method comprising intravesical administration of a formulation comprised of microparticles having a mean diameter of 50 nm to 2000 μm, 2.5 wt. % to 95 wt. % of a pharmacologically active agent, and 5 wt. % to 97.5 wt. % of a controlled release carrier,
wherein the microparticles are buoyant in urine, and
wherein the formulation is at least partially retained by the bladder for a duration of a drug delivery time period upon which the active agent is released.

36. The method of claim 35, wherein the microparticles have a mean specific gravity of less than 1.03.

37. (canceled)

38. The method of claim 35, wherein the pharmacologically active agent comprises an anti-infective agent, an anesthetic agent, an analgesic agent, a diuretic, an anti-inflammatory agent, a coagulant or an anti-coagulant, a chemotherapeutic agent, an agent for the treatment of incontinence, an RAAS inhibitor, an agent for treating kidney stones, a contrast agent for diagnostics and monitoring, or a combination thereof.

39. (canceled)

40. The method of claim 39, wherein the urinary system disorder is an infection, cancer, incontinence, a kidney stone or a urinary tract infection.

41.-51. (canceled)

52. The method of claim 35, wherein the controlled release comprises (a) sustained release, (b) bolus dose followed by sustained release or (c) zero order release.

53.-57. (canceled)

58. The method of claim 35, wherein a unit dose of the formulation is administered to the subject, the unit dose comprising 60 mg to 5 g of the pharmacologically active agent.

59.-67. (canceled)
